(19) United States
(12) Patent Application Publication
(10) Pub. No.: US 2010/0168129 A1
(43) Pub. Date: Jul. 1, 2010

(54) DRUGS WITH ANTICHOLESTATIC ACTIVITY

(76) Inventors: Mario Brufani, Castel Gandolfo (IT); Luciano Cellai, Roma (IT); Barbara Bartolini, Roma (IT); Ilaria Medici, Roma (IT); Bianca Maria Lagrasta, Roma (IT)

Correspondence Address: COLLARD & ROE, P.C., 1077 NORTHERN BOULEVARD, ROSLYN, NY, 11576, US

(21) Appl. No.: 12/452,674

(22) PCT Filed: Jul. 17, 2008
(86) PCT No.: PCT/EP2008/059376

§ 371 Date: Jan. 14, 2010

(30) Foreign Application Priority Data
Jul. 17, 2007 (IT) MI2007A001435

(51) Int. Cl. A61K 31/496; C07D 491/048; A61P 3/00

(52) U.S. Cl. 514/254.11; 540/458

(57) ABSTRACT

New compounds belonging to the structural formula (I) are described.

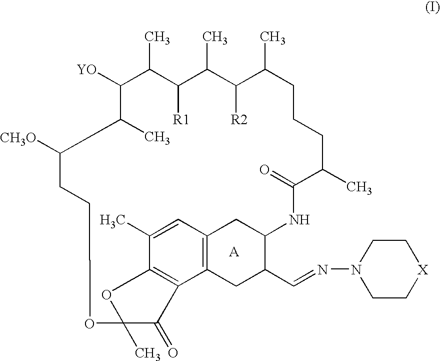

in which R1, R2, A, Y and X are specified in the description, useful in the treatment of cholestasis and substantially devoid of antibacterial activity. The synthesis process of said compounds, the pharmaceutical compositions containing them and their use in therapy are also described.

### FIELD OF THE INVENTION

The present invention relates to the field of drugs effective in the treatment of cholestasis and diseases related thereto.

### STATE OF THE ART

The use of various rifamycin derivatives in antibiotic therapy is known; in particular rifampicin in the SV form (see structure on the left) is used, and can be oxidized into the S form (see structure on the right).

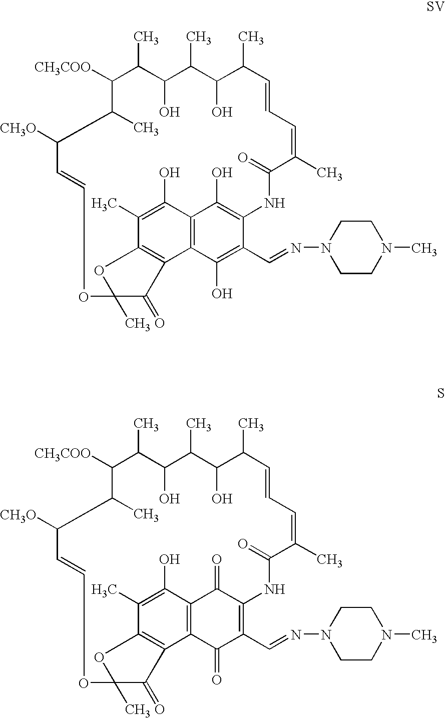

In particular, rifampicin has proved to be useful in the treatment of tuberculosis and leprosy.

Rifampicin has also been the subject of research with regard to possible additional biological effects. Certain studies have evaluated its effect on bile acid metabolism, but contrasting results were obtained. For example, J. Lipid Res., 2002, 43, pp 359-364 reports the potential usefulness of rifampicin in the treatment of biliary cholestasis, while other works (see for example Ann. Hepathol., 2003, 2(4), p. 150-158, and Ann. Gastroenterol., 2001, 14(4), 281-87) relate that treatment with rifampicin and rifamycin SV is itself the cause of intrahepatic cholestasis. Cholestasis is caused by a functional defect in the formation of bile in the hepatocytes or by a reduction in the secretion and flow of bile in the biliary duct. Intrahepatic cholestasis is mainly due to the inability of hepatocytes to secrete bile; extrahepatic cholestasis is caused by obstruction of the drainage system consisting of the bile duct. Certain studies have established that rifampicin is an agonist of human nuclear receptor PXR, whose activation induces transcription of the genes coding for cytochrome CYP3A4 and other enzymes involved in the metabolism and transport of bile acids (J. Lipid Res., 2002, 43, pag. 359-364) and inhibits transcription of the CYP7A1 gene, which codes for cholesterol 7α-hydroxylase, being the enzyme responsible for the first step in the transformation of cholesterol into primary bile acids (Am. J. Physiol. Gastrointest Liver Physiol 288: G74-G84, 2005).

Certain derivatives of the aforesaid structures are known, whose chemical modifications include the reduction of specific double bonds. For example, Eur. J. Biochem., 1975, 52, 391-400 describes the antibacterial activity of some derivatives of hexahydrorifamycin S and SV. U.S. Pat. No. 4,261,891 describes the antibacterial activity of a family of rifamycin hexahydroderivatives, substituted in position 3 with an azacycloalkyl ring.

In another work (Biochim. Biophys. Acta., 1969, 182, 24-29), the same activity was evaluated for the 23,27 epoxyderivative of rifamycin SV.

U.S. Pat. No. 4,017,481 describes compounds of formula

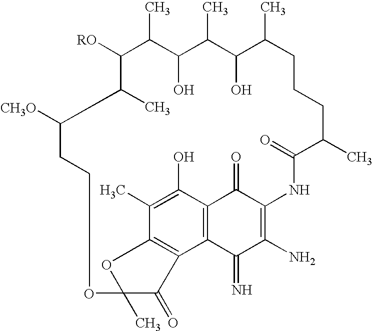

as being powerful antimicrobial agents.

### SUMMARY

It has now been discovered that the compounds belonging to the structural formula (I)

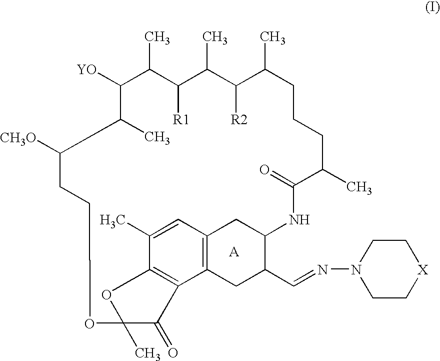

where:
R₁ and R₂ are chosen from OH or OCH₃, or R₁ and R₂ taken together form a —O—C(CH₃)₂—O— group, the ring A is chosen from:

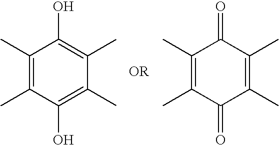

Y is chosen from H and CO—CH₃,
X is chosen from: CH₂, O, S, NH, NR₃,N—COR₃, where R₃ represents:
a) a linear or branched alkyl group,
b) a (CH₂)ₙ—R₄ chain where n is comprised between 0 and 8, and R4 is chosen from OH, NH₂, halogen, a cycloalkyl, aryl or heterocyclic group,
c) a (CH₂)ₘ—Z—(CH₂)ₙCH₃ chain where m+n is comprised between 1 and 8, and Z represents —O—, —S—, —NH—, —N(R₅) where R₅ is a linear or branched alkyl,
are surprisingly highly active in activating the PXR receptor, and are therefore useful in the treatment of cholestasis. The compounds of formula (I) have also been shown to be substantially devoid of antibacterial activity, this enabling their prolonged use without any risk of giving rise to resistant bacterial strains.

### DESCRIPTION OF THE FIGURES

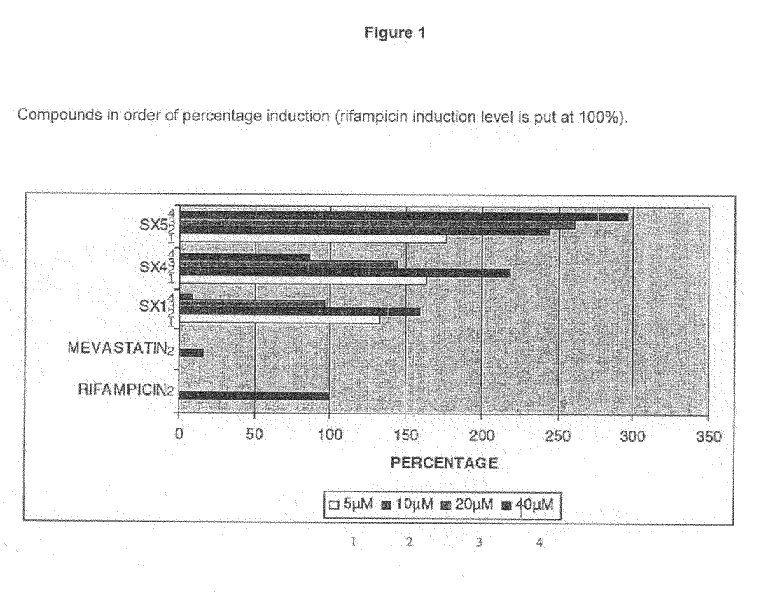

The Invention will be now described in details in the following with reference to the FIG. 1, wherein the results of the experimental part of the preset invention are shown.

### DETAILED DESCRIPTION

The aforesaid compounds of formula (I), being new, are themselves a first aspect of the invention. In formula (I) the carbon atom in position 16 is chiral: formula (I) hence comprises without distinction the corresponding isolated epimers, the equivalent epimer mixtures and the mixtures enriched with one or the other epimer.

In formula (I) all the alkyl groups can be linear or branched and preferably contain from 1 to 9 carbon atoms; specific examples of alkyl groups are: ethyl, propyl, isopropyl, butyl, isobutyl, sec-butyl, tert-butyl, n-pentyl, 2-methyl-butyl, n-hexyl, 2-methyl-pentyl and so forth. All the cycloalkyl groups contain from 5 to 8 members. The said heterocyclic groups can be aromatic or non aromatic and contain from 5 to 8 members, including one or more heteroatoms chosen from N, O, S. All the said cycloalkyl, aryl, heterocyclic groups can be substituted or non substituted with groups preferably chosen from halogen, hydroxyl, C₁₋₄ alkyl, C₁₋₄ alkoxy. In the case of the R₃ radical, the aforesaid condition “m+n comprised between 1 and 8” includes the possibility that one from m and n=0. According to a preferred embodiment of the invention, X is NR₃. In particular R₃ preferably represents:
a C₁₋₄ alkyl,
a (CH₂)ₙ—R₄ chain where n is 0 and R₄ is chosen from cyclohexyl, phenyl, piperidino, morpholino and thiomorpholino,
a (CH₂)ₘ—Z—(CH₂)ₙ—CH₃ chain with m+n comprised between 1 and 5, and Z representing —O—.

Particularly preferred compounds of formula (I) are the following:
3-(4′-methyl-1′-piperazinyl-iminomethyl)-16,17,18,19,28,29-hexahydrorifamycin SV
3-(4′-methyl-1′-piperazinyl-iminomethyl)-21,23-O-isopropylidene-16,17,18,19,28,29-hexahydrorifamycin SV
3-(4′-methyl-1′-piperazinyl-iminomethyl)-16,17,18,19,28,29-hexahydrorifamycin S
3-(4′-methyl-1′-piperazinyl-iminomethyl)-21,23-O-isopropylidene-16,17,18,19,28,29-hexahydrorifamycin S
3-(4′-ethyl-1′-piperazinyl-iminomethyl)-16,17,18,19,28,29-hexahydrorifamycin SV
3-(4′-ethyl-1′-piperazinyl-iminomethyl)-21,23-O-isopropylidene-16,17,18,19,28,29-hexahydrorifamycin SV
3-(4′-ethyl-1′-piperazinyl-iminomethyl)-16,17,18,19,28,29-hexahydrorifamycin S
3-(4′-ethyl-1′-piperazinyl-iminomethyl)-21,23-O-isopropylidene-16,17,18,19,28,29-hexahydrorifamycin S
3-(1′-piperidinyl-iminomethyl)-16,17,18,19,28,29-hexahydrorifamycin SV
3-(1′-piperidinyl-iminomethyl)-21,23-O-isopropylidene-16,17,18,19,28,29-hexahydrorifamycin SV
3-(1′-piperidinyl-iminomethyl)-16,17,18,19,28,29-hexahydrorifamycin S
3-(1′-piperidinyl-iminomethyl)-21,23-O-isopropylidene-16,17,18,19,28,29-hexahydrorifamycin S
3-(4′-phenyl-1′-piperazinyl-iminomethyl)-16,17,18,19,28,29-hexahydrorifamycin SV
3-(4′-phenyl-1′-piperazinyl-iminomethyl)-21,23-O-isopropylidene-16,17,18,19,28,29-hexahydrorifamycin SV
3-(4′-phenyl-1′-piperazinyl-iminomethyl)-16,17,18,19,28,29-hexahydro-rifamycin S
3-(4′-phenyl-1′-piperazinyl-iminomethyl)-21,23-O-isopropylidene-16,17,18,19,28,29-hexahydrorifamycin S
3-[4′-(2-ethoxyethyl)-1′-piperazinyl-iminomethyl]-16,17,18,19,28,29-hexahydrorifamycin SV
3-[4′-(2-ethoxyethyl)-1′-piperazinyl-iminomethyl]-21,23-O-isopropylidene-16,17,18,19,28,29-hexahydrorifamycin SV
3-[4′-(2-ethoxyethyl)-1′-piperazinyl-iminomethyl]-16,17,18,19,28,29-hexahydrorifamycin S
3-[4′-(2-ethoxyethyl)-1′-piperazinyl-iminomethyl]-21,23-O-isopropylidene-16,17,18,19,28,29-hexahydrorifamycin S
3-(1′-morpholinyl-iminomethyl)-16,17,18,19,28,29-hexahydrorifamycin SV
3-(1′-morpholinyl-iminomethyl)-21,23-O-isopropylidene-16,17,18,19,28,29-hexahydrorifamycin SV
3-(1′-morpholinyl-iminomethyl)-16,17,18,19,28,29-hexahydrorifamycin S
3-(1′-morpholinyl-iminomethyl)-21,23-O-isopropylidene-16,17,18,19,28,29-hexahydrorifamycin S
3-(t-thiomorpholinyl-iminomethyl)-16,17,18,19,28,29-hexahydrorifamycin SV
3-(1′-thiomorpholinyl-iminomethyl)-21,23-O-isopropylidene-16,17,18,19,28,29-hexahydrorifamycin SV
3-(1′-thiomorpholinyl-iminomethyl)-16,17,18,19,28,29-hexahydrorifamycin S
3-(1′-thiomorpholinyl-iminomethyl)-21,23-O-isopropylidene-16,17,18,19,28,29-hexahydrorifamycin S
3-(4′-isopropyl-1′-piperazinyl-iminomethyl)-16,17,18,19,28,29-hexahydrorifamycin SV
3-(4′-isopropyl-1′-piperazinyl-iminomethyl)-21,23-O-isopropylidene-16,17,18,19,28,29-hexahydrorifamycin SV
3-(4′-isopropyl-1′-piperazinyl-iminomethyl)-16,17,18,19,28,29-hexahydrorifamycin S
3-(4′-isopropyl-1′-piperazinyl-iminomethyl)-21,23-O-isopropylidene-16,17,18,19,28,29-hexahydrorifamycin S
3-(4′-cyclohexyl-1′-piperazinyl-iminomethyl)-16,17,18,19,28,29-hexahydrorifamycin SV
3-(4′-cyclohexyl-1′-piperazinyl-iminomethyl)-21,23-O-isopropylidene-16,17,18,19,28,29-hexahydrorifamycin SV
3-(4′-cyclohexyl-1′-piperazinyl-iminomethyl)-16,17,18,19,28,29-hexahydrorifamycin S
3-(4′-cyclohexyl-1′-piperazinyl-iminomethyl)-21,23-O-isopropylidene-16,17,18,19,28,29-hexahydrorifamycin S
3-(4′-methyl-1′-piperazinyl-iminomethyl)-16,17,18,19,28,29-hexahydro-25-desacetylrifamycin SV
3-[4′-(2-hydroxyethyl)-1′-piperazinyl-iminomethyl]-16,17,18,19,28,29-hexahydrorifamycin SV

A further aspect of the invention is a process for preparing the aforedefined compounds of formula (I); in its general meaning the process starts from rifamycin (S or SV) and comprises the following steps:

(i) reduction of the double bonds in positions 16,17,18,19,28,29
(ii) addition of the group

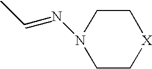

in position 3.

Step (i) is preferably carried out by hydrogenation of rifamycin (S or SV) in a protic solvent (e.g. EtOH) and in the presence of a catalytic activator of hydrogenation, e.g. platinum oxide; independently of the starting form of rifamycin (S or SV), the intermediate product of formula (II) is obtained:

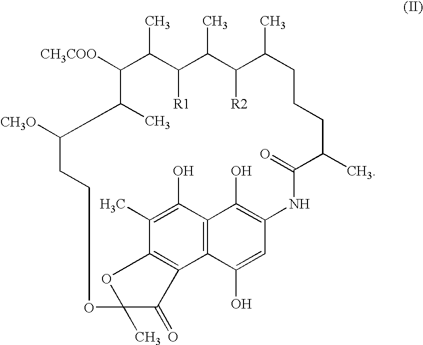

The derivative (II) can be treated with formaldehyde and a primary amine, e.g. t-butylamine, in the presence of an oxidizing agent, e.g. manganese dioxide; the reaction is undertaken for a time comprised between 5 and 20 hours, preferably 12 hours, at a temperature comprised between 30 and 70° C., preferably 50° C. After being filtered and dried, the crude reaction product is used without further purification and reacted with a hydrazine of formula

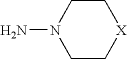

where X has the aforedefined meanings for formula (I). The final compound of formula (I) is obtained in the form of a mixture of the two forms (S+SV). The desired compound (S or SV) is isolatable from this mixture by means of commonly known methods such as column chromatography.

Before carrying out the separation, the S+SV mixture can be enriched in the desired form (S or SV) by treatment with oxidizing or reducing agents respectively: suitable oxidants are potassium ferricyanide, nitrous acid, or manganese dioxide; a suitable reducing agent is ascorbic acid. The same oxidants/reducers are also usable downstream of the separation by converting SV into S or vice-versa.

Should compounds of formula (I) be required in which R₁ and R₂ together form the —O—C(CH₃)₂—O— group, in addition to the aforementioned steps, and before step (i), the S or SV rifamycin is treated with acetone dimethylketal to derivatize the C(21) and C(23). The derivative obtained then follows the same synthesis path aforedescribed for S or SV rifamycin, obtaining at the end the compound (I) in which R1 and R2 together form the —O—C(CH₃)₂—O— group.

Should compounds of formula (I) in which Y═H be required, the synthesis process starts from the corresponding C₂₅—O-desacetylate of S or SV rifamycin.

The compounds according to the aforesaid formula (I) possess a high capacity for activating receptor PXR, and therefore can be used in the prevention and treatment of all pathological conditions related to cholestasis.

As seen experimentally, the activity was found to be from 1.5 to 3 times greater than that of rifampicin. This result was not in any way foreseeable. In particular, reduction of the double bonds contained in the alkylene loop of rifampicin has led to a drastic increase in activation of the PXR receptor involved in the therapeutic response to cholestasis. At the same time, the antibacterial activity was found to be substantially absent: for this reason the new compounds, being already effective at low doses by virtue of their increased activity, can be used for long periods without the danger of resistant bacterial strains arising.

The invention therefore comprises the use of one or more compounds of formula (I) as aforedefined in the preparation of a useful drug for the prevention and/or treatment of cholestasis and of diseases related thereto, in man or in animals.

Examples of such conditions include: obstructive cholestasis, drug induced cholestasis, Dubin-Johson Syndrome, sitosterolemia and in general all hepatobiliary transport disorders.

The invention further extends to the aforedefined compounds of formula (I) for use in therapy, in particularly in the treatment of cholestasis and diseases related thereto, as exemplified above.

A further aspect of the invention is a method for the prevention and/or treatment of cholestasis, characterized by the administration, to a patient requiring it, of a pharmaceutically effective quantity of one or more compounds of formula (I).

In the aforesaid uses and methods, the dosage of the compounds of formula (I) can vary according to the type and condition of the patient, the degree of disease severity, the chosen administration route and the number of daily administrations carried out, etc. As an indication, they can be administered at a dosage range comprised between 1 and 100 mg/kg/day.

The compounds can be used alone, or co-administered with other pharmaceutical therapies having cholestasis or the risk thereof as a secondary effect. Administration is undertaken by means of suitable pharmaceutical compositions, produced according to known techniques.

The invention hence comprises new pharmaceutical compositions characterized by containing one or more active principles of formula (I) in combination with excipients and pharmaceutically acceptable diluents.

Said compositions are prepared by blending of the relative components and are suitably adapted to oral or parenteral administration, and as such can be administered in the form of tablets, capsules, oral preparations, powders, granules, pills, injectable or infusible liquid solutions or suspensions or suppositories.

Tablets and capsules for oral administration are normally presented in unit dose form and contain conventional excipients such as binders, fillers, diluents, compaction agents, lubricants, detergents, disintegrants, colouring agents, flavouring agents and wetting agents. The tablets can be coated according to methods well known in the art.

Suitable fillers include cellulose, mannitol, lactose and other similar agents. Suitable disintegrants include starch, polyvinylpyrrolidone and starch derivatives such as sodium glycolate starch. Suitable lubricants include, for example, magnesium stearate. Suitable wetting agents include sodium lauryl sulfate.

These solid oral compositions can be prepared by conventional methods of blending, filling or compaction. Oral liquid preparations can be in the form of, for example, aqueous or oily suspensions, solutions, emulsions, syrups or elixirs, or can be presented as a dry product for reconstitution with water or with a suitable vehicle before use. Such liquid preparations can contain conventional additives such as suspending agents, for example sorbitol, syrup, methyl cellulose, gelatin, hydroxyethyl cellulose, carboxymethyl cellulose, aluminium stearate gel, or hydrogenated edible fats; emulsifying agents, such as lecithin, sorbitan monooleate, or acacia; non-aqueous vehicles (which can include edible oils), such as almond oil, fractionated coconut oil, oily esters such as esters of glycerine, propylene glycol, or ethyl alcohol; preservatives, such as methyl or propyl p-hydroxybenzoate or ascorbic acid, and if desired, conventional flavouring or colouring agents.

Oral formulations also include conventional retard release formulations such as enterically coated tablets or granules.

For parenteral administration, fluid dosage units can be prepared, containing the compound and a sterile vehicle. The compound can be either suspended or dissolved, depending on the vehicle and concentration. The parenteral solutions are normally prepared by dissolving the compound in a vehicle and filter sterilizing before filling suitable vials or ampoules and sealing them. Advantageously, adjuvants such as local anaesthetics, preservatives and buffering agents can also be dissolved in the vehicle. To increase its stability, the composition can be frozen after having filled the vials and removed the water under vacuum. Parenteral suspensions are prepared in substantially the same manner, except that the compound can be suspended in the vehicle instead of being dissolved, and sterilized by exposure to ethylene oxide before suspending in the sterile vehicle. Advantageously, a surfactant or wetting agent can be included in the composition to facilitate uniform distribution of the compound of the invention.

Another means of administering the compounds of the invention concerns a topical treatment. Topical formulations can contain for example ointments, creams, lotions, gels, solutions, pastes and/or can contain liposomes, micelles and/or microspheres. Examples of ointments include oleaginous ointments such as vegetable oils, animal fats, semisolid hydrocarbons; emulsifiable ointments such as hydroxystearin sulphate, anhydrous lanolin, hydrophilic petrolatum, cetyl alcohol, glycerol monostearate, stearic acid; water soluble ointments containing polyethylene glycols of various molecular weights. A reference for the formulations is the book by Remington (“Remington: The Science and Practice of Pharmacy”, Lippincott Williams & Willcins, 2000). Creams, as known to formulation experts, are viscous liquids or semisolid emulsions, and contain an oil phase, an emulsifier and an aqueous phase. The oil phase generally contains petrolatum and an alcohol such as cetyl or stearic alcohol. The emulsifier in a cream formulation is chosen from non-ionic, anionic, cationic or amphoteric surfactants. The monophasic gels contain organic macro-molecules uniformly distributed in the liquid, which is generally aqueous, but they also preferably contain an alcohol and optionally an oil. Preferred gelling agents are cross-linked acrylic acid polymers (e.g. carbomer-type polymers, such as carboxypolyalkylenes, which are commercially available under the Carbopol™ trademark). Hydrophilic polymers are also preferred, such as polyoxyethylene, polyoxyethylene-polyoxypropylene copolymers and polyvinyl alcohol; cellulose polymers such as hydroxypropyl cellulose, hydroxyethyl cellulose, hydroxypropyl methylcellulose, hydroxypropyl methylcellulose phthalate and methylcellulose; gums, such as xanthan gum and tragacanth gum; sodium alginate; and gelatin. Dispersing agents such as alcohol or glycerin can be added for gel preparation. The gelling agent can be dispersed by chopping and/or mixing.

A further method of administering the compounds of the invention concerns transdermal delivery. Typical transdermal formulations comprise conventional aqueous and non-aqueous vectors, such as creams, oils, lotions or pastes or can be in the form of membranes or medicated patches. One formulation provides that a compound of the invention is dispersed within a pressure sensitive patch which adheres to the skin. This formulation enables the compound to diffuse from the patch to the patient through the skin. For a constant release of the drug through the skin, natural rubber and silicon can be used as pressure sensitive adhesives. As is common practice, the compositions are normally accompanied by written or printed instructions for use in the treatment in question.

The following non-limiting examples serve to illustrate the invention.

### EXPERIMENTAL PART

### Example 1

### 3-(4′-methyl-1′-piperazinyl-iminomethyl)-16,17,18,19,28,29-hexahydrorifamycin SV

3 g of S or SV rifamycin are dissolved in 150 ml of EtOH to which 500 mg of PtO₂ are added. The mixture is left under H₂ atmosphere for 4 hours under agitation. It is then filtered over celite and concentrated. 6 ml of 0.3 M NaNO₂ are added then 2 M HCl until an acidic pH is achieved. Water is added and the mixture extracted with CHCl₃; the organic phase is washed with saturated NaCl solution, dried over anhydrous Na₂SO₄, filtered and concentrated to dryness.

The product is purified using a column packed with 300 g of 200-425 mesh silica with 100% CHCl₃ and after loading, a gradient is performed with AcOEt to a 8:2 ratio.

2.15 g of 16,17,18,19,28,29 hexahydrorifamycin S is obtained with Rf=0.47 on TLC in 8:2 CHCl₃:AcOEt.

By way of the same synthesis path, but eliminating treatment with 0.3 M NaNO₂ and HCl, 16,17,18,19,28,29 hexahydrorifamycin SV is obtained with Rf=0.4 on TLC in 1:1 CHCl₃:AcOEt.

2 g of 16,17,18,19,28,29 hexahydrorifamycin S or SV obtained in this manner are dissolved in 50 ml of THF; then 1.27 ml of t-butylamine, 530 μl of 37% formaldehyde and 1.3 g of MnO₂ are added. The reaction is allowed to proceed overnight under agitation at 50° C. The mixture is filtered over celite to remove MnO₂ and concentrated. Water is added and the mixture extracted with CHCl₃; the organic phase is subsequently washed with saturated NaCl solution, dried over anhydrous Na₂SO₄, filtered and concentrated to dryness.

The crude reaction product is dissolved in 50 ml THF and 820 μl of 1-amino-4-methylpiperizine (commercial) are added. The mixture is allowed to react for 4-5 hours under agitation at ambient temperature, then concentrated. Water is added and the mixture extracted with CHCl₃; the organic phase is subsequently washed with saturated NaCl solution, dried over anhydrous Na₂SO₄, filtered and concentrated to dryness.

The product is purified using a column packed with 150 g of 200-425 mesh silica in 1:1 CHCl₃/AcOEt and eluted with 1:1 CHCl₃/AcOEt adding EtOH at a 1% to 10% gradient.

200 mg of 3-(4′-methyl-1′-piperazinyl-iminomethyl)-16,17,18,19,28,29-hexahydrorifamycin SV are obtained with Rf=0.290 on TLC in 1:1 CHCl₃/AcOEt with 10% EtOH.

¹H-NMR (CDCl₃) 400 MHz: −0.3 (d, 3H); 0.5 (d, 3H); 0.7 (d, 3H); 1.0 (d, 3H); 1.3 (d, 3H); 1.2-1.4 (m, total 6H); 1.7 (s, 3H); 2.0 (s, 3H); 2.2 (s, 3H); 2.4 (s, 3H); 2.6 (m, 4H); 2.9 (m, 1H); 3.2 (m, 4H); 3.4-3.6 (m, 3H); 5 (d, 1H); 8.3 (s, 1H); 12.2 (s, 1H).

### Example 2

### 3-(4′-methyl-1′-piperazinyl-iminomethyl)-16,17,18,19,28,29-hexahydrorifamycin S

The product 3-(4′-methyl-1′-piperazinyl-iminomethyl)-16,17,18,19,28,29-hexahydrorifamycin SV, synthesized as described in example 1 is dissolved in CHCl₃ and shaken with an aqueous 33% potassium ferricyanide solution. The oxidation product is extracted with CHCl₃, the organic phase is washed with saturated NaCl solution, dried over anhydrous Na₂SO₄, filtered and concentrated to dryness.

The product 3-(4′-methyl-1′-piperazinyl-iminomethyl)-16,17,18,19,28,29-hexahydrorifamycin S has an Rf=0.294 on TLC in 1:1 CHCl₃/AcOEt with 10% EtOH.

¹H-NMR (CDCl₃) 400 MHz: 0.3 (d, 3H); 0.5 (d, 3H); 0.7 (d, 3H); 1.0 (d, 3H); 1.3 (d, 3H); 1.4-1.6 (m, total 6H); 1.7 (s, 3H); 2.1 (s, 3H); 2.2 (s, 3H); 2.4 (s, 3H); 2.6 (m, 4H); 3 (m, 1H); 3.3 (m, 4H); 3.6-3.8 (m, 4H); 5 (d, 1H); 8.4 (s, 1H); 12.2 (s, 1H).

### Example 3

### 3-(4′-methyl-1′-piperazinyl-iminomethyl)-21,23-O-isopropylidene-16,17,18,19,28,29-hexahydrorifamycin SV

3.2 ml of dimethylketal acetone and 125 μl of a solution of 0.5 ml of conc. H₂SO₄ in 20 ml of acetone are added to 3.2 g of rifamycin S in 32 ml of acetone.

The mixture is left for 1 hour under agitation at ambient temperature then Na₂CO₃ is added to neutralize the solution. It is filtered through paper, and concentrated to dryness.

The product is purified using a column packed with 90 g of 100-200 mesh silica and eluted with 85:15 CHCl₃/AcOEt. About 2.5 g of 21,23-O-isopropylidene rifamycin S are obtained. The product has a Rf=0.5 on TLC in 85:15 CHCl₃/AcOEt.

2.5 g of 21,23-O-isopropylidene rifamycin S (or SV, obtained by reduction with an aqueous ascorbic acid solution according to the process described in detail in example 6) obtained in this manner are dissolved in 200 ml of EtOH and 500 mg of PtO₂ are added. The mixture is left under H₂ atmosphere for 4 hours under agitation. It is then filtered over celite and concentrated. 6 ml of 0.3 M NaNO₂ are added then 2M HCl until an acidic pH is achieved. Water is added and the mixture extracted with CHCl₃; the organic phase is washed with saturated NaCl solution, dried over anhydrous Na₂SO₄, filtered and concentrated to dryness.

The product is purified using a column packed with 200 g of 200-425 mesh silica and flushed with 9:1 CHCl₃/AcOEt. 1.76 g of 21,23-O-isopropylidene-16,17,18,19,28,29-hexahydrorifamycin S are obtained. The product has a Rf=0.76 on TLC in 9:1 CHCl₃/AcOEt.

1.76 g of 21,23-O-isopropylidene-16,17,18,19,28,29-hexahydrorifamycin S or SV are dissolved in 35 ml of THF; 1.05 ml of t-butylamine, 443 μl of 37% formaldehyde and 1.05 g of MnO₂ are added. The mixture is reacted overnight under agitation at 50° C. The mixture is filtered over celite to remove MnO₂ and concentrated. Water is added and the mixture extracted with CHCl₃; the organic phase is subsequently washed with saturated NaCl solution, dried over anhydrous Na₂SO₄, filtered and concentrated to dryness.

The reaction mixture, evaporated under vacuum, is re-dissolved in 35 ml of THF and 549 μl of 1-amino-4-methylpiperazine are added. The mixture is allowed to react for 4-5 hours under agitation at ambient temperature, then concentrated. Water is added and the mixture extracted with CHCl₃; the organic phase is subsequently washed with saturated NaCl solution, dried over anhydrous Na₂SO₄, filtered and concentrated to dryness.

The product is purified using a column packed with 51 g of 200-425 mesh silica in 1:1 CHCl₃/AcOEt and eluted with 1:1 CHCl₃/AcOEt adding EtOH at a 1%-10% gradient.

704 mg of 3-(4′-methyl-1′-piperazinyl-iminomethyl)-21,23-O-isopropylidene-16,17,18,19,28,29-hexahydrorifamycin SV with Rf=0.54 on TLC in 9:1 CHCl₃:EtOH.

¹H-NMR (CDCl₃) 400 MHz: −0.02 (d, 3H); 0.6 (d, 3H); 0.7 (d, 3H); 0.9 (d, 3H); 1.2 (dd, 6H); 1.5-2.0 (m, 5H); 1.8 (s, 3H); 2.0 (s, 3H); 2.2 (s, 3H); 2.4 (s, 3H); 2.8 (s, 4H); 2.4 (s, 4H); 2.8-3.0 (m, 3H); 3.2 (s, 3H); 3.4 (m, 1H); 5.2 (dd, 1H); 8.2 (s, 1H); 12.0 (s, 1H).

### Example 4

### 3-(4′-methyl-1′-piperazinyl-iminomethyl)-21,23-O-isopropylidene-16,17,18,19,28,29-hexahydrorifamycin S

The product 3-(4′-methyl-1′-piperazinyl-iminomethyl)-21,23-O-isopropylidene-16,17,18,19,28,29-hexahydrorifamycin SV, synthesized as described in example 3 is dissolved in CHCl₃ and shaken with an aqueous 33% potassium ferricyanide solution. The oxidation product is extracted with CHCl₃, the organic phase is washed with saturated NaCl solution, dried over anhydrous Na₂SO₄, filtered and concentrated to dryness.

The product 3-(4′-methyl-1′-piperazinyl-iminomethyl)-21,23-O-isopropylidene-16,17,18,19,28,29-hexahydrorifamycin S has a Rf=0.8 on TLC in 9:1 CHCl₃:EtOH.

¹H-NMR (CDCl₃) 400 MHz: 0.6 (d, 3H); 0.7 (d, 3H); 0.8 (d, 3H); 0.82 (d, 3H); 1.2 (dd, 6H); 1.4-1.55 (m, total 6H); 1.6 (s, 3H); 1.8 (s, 3H); 2.3 (s, 3H); 2.4 (s, 3H); 2.6 (m, 4H); 2.9-3.0 (m, total 2H); 3.3 (s, 3H); 3.35 (m, 4H); 3.6 (m, 1H); 4.9 (d, 1H); 7.8 (s, 1H); 11.0 (s, 1H).

### Example 5

### 3-(4′-ethyl-1′-piperazinyl-iminomethyl)-16,17,18,19,28,29-hexahydrorifamycin S

510 mg of NaNO₂ previously dissolved in 670 μl of H₂O are added to a solution of 860 mg of 1-ethylpiperazine in 5.59 ml of H₂O over 1 hour. The mixture is then acidified with 36% HCl and left under agitation for 20 minutes. The solution is then treated with NaOH to neutral pH and extracted with CHCl₃. The organic phase is dried over anhydrous Na₂SO₄, filtered and concentrated to dryness. 860 mg of 1-nitroso-4-ethylpiperazine are obtained as a yellow liquid. Rf=0.58 in TLC with 9:1 CHCl₃/MeOH.

This compound is then dissolved in acetic acid and water (1:1, v/v), and 1.29 g of powdered Zn are added over 20 minutes. When additions are completed, the mixture is heated to 50° C. for 1 hour, then filtered and 7.61 ml of 50% NaOH are added to the solution. The formation of a white emulsion is observed. The mixture is extracted with CHCl₃, the organic phase is dried over Na₂SO₄, filtered and concentrated to dryness. 735 mg of 1-amino-4-ethylpiperazine are obtained. 2.0 g of 16,17,18,19,28,29-hexahydrorifamycin S or SV, prepared as described in example 1 are dissolved in 50 ml of THF; 1.27 ml of t-butylamine, 530 μl of 37% formaldehyde and 1.3 g of MnO₂ are then added. The reaction is allowed to proceed overnight under agitation at 50° C. It is filtered over celite to remove MnO₂ and concentrated. Water is added and the mixture extracted with CHCl₃; the organic phase is subsequently washed with saturated NaCl solution, dried over anhydrous Na₂SO₄, filtered and concentrated to dryness.

The crude reaction product is dissolved in 50 ml of THF and 735 mg of 1-amino-4-ethylpiperazine are added. The reaction is left for 4-5 hours under agitation at ambient temperature; water is added and the mixture extracted with CHCl₃; the organic phase is subsequently washed with saturated NaCl solution, dried over anhydrous Na₂SO₄, filtered and concentrated to dryness.

The product is purified using a column packed with 150 g of 200-425 mesh silica in 1:1 CHCl₃/AcOEt and eluted with 1:1 CHCl₃/AcOEt adding EtOH at a 1%-10% gradient.

150 mg of 3-(4′-ethyl-1′-piperazinyl-iminomethyl)-16,17,18,19,28,29-hexahydrorifamycin S are obtained with Rf=0.33 on TLC in 1:1 CHCl₃/EtOH with 10% EtOH.

¹H-NMR (CDCl₃) 400 MHz: 0.24 (d, 3H); 0.3 (d, 3H); 0.65 (d, 3H); 0.8 (d, 3H); 1.14 (t, 3H); 1.2 (d, 3H); 1.5-1.85 (m, total 6H); 1.8 (s, 3H); 2.08 (s, 3H); 2.25 (s, 3H); 2.5 (q, 2H); 2.65 (m, 4H); 3.0-3.8 (m, total 5H); 3.17 (s, 3H); 3.41 (m, 6H); 3.6 (m, 2H); 5.01 (d, 1H); 7.75 (s, 1H); 10.85 (s, 1H).

### Example 6

### 3-(4′-ethyl-1′-piperazinyl-iminomethyl)-16,17,18,19,28,29-hexahydrorifamycin SV

200 mg of 3-(4′-ethyl-1′-piperazinyl-iminomethyl)-16,17,18,19,28,29-hexahydrorifamycin S synthesized as described in example 5 are dissolved in a water-miscible organic solvent (e.g. MeOH or acetone) and an aqueous 15% ascorbic acid solution is then added. The reaction mixture is concentrated under vacuum, the reduction product is extracted with CHCl₃; the organic phase is washed with saturated NaCl solution, dried over anhydrous Na₂SO₄, filtered and concentrated to dryness.

The product is purified using a column packed with 15 g of 200-425 mesh silica in 1:1 CHCl₃/AcOEt and eluted with 1:1 CHCl₃/AcOEt adding EtOH at a 1%-10% gradient.

125 mg of 3-(4′-ethyl-1′-piperazinyl-iminomethyl)-16,17,18,19,28,29-hexahydrorifamycin SV are obtained with Rf=0.44 on TLC in 1:1 CHCl₃/AcOEt with 10% of EtOH.

¹H-NMR (CDCl₃) 400 MHz: −0.3 (d, 3H); 0.5 (d, 3H); 0.62 (d, 3H); 1.0 (d, 3H); 1.2 (t, 3H); 1.4 (d, 2H); 1.4-2.1 (m, total 6H); 1.7 (s, 3H); 2.05 (s, 3H); 2.12 (s, 3H); 2.5 (q, 2H); 2.7 (m, 4H); 3.0 (m, 1H); 3.02 (s, 3H); 3.3 (m, 4H); 3.3-3.8 (m, total 4H); 4.9 (d, 1H); 8.21 (s, 1H); 12.05 (s, 1H).

### Example 7

### 3-(1′-piperidinyl-iminomethyl)-16,17,18,19,28,29-hexahydrorifamycin S

2.0 g of 16,17,18,19,28,29-hexahydrorifamycin S or SV, prepared as described in example 1 are dissolved in 50 ml of THF; 1.27 ml of t-butylamine, 530 μl of 37% formaldehyde and 1.3 g of MnO₂ are then added. The reaction is allowed to proceed overnight under agitation at 50° C. It is filtered over celite to remove MnO₂ and concentrated. Water is added and the mixture extracted with CHCl₃; the organic phase is subsequently washed with saturated NaCl solution, dried over anhydrous Na₂SO₄, filtered and concentrated to dryness.

The crude reaction product is dissolved in 50 ml of THF and 285.4 μl of N-aminopiperidine (commercial) are added. The reaction is left for 4-5 hours under agitation at ambient temperature and concentrated; water is added and the mixture extracted with CHCl₃; the organic phase is subsequently washed with saturated NaCl solution, dried over anhydrous Na₂SO₄, filtered and concentrated to dryness.

The product is purified using a column packed with 150 g of 200-425 mesh silica in 1:1 CHCl₃/AcOEt and eluted with 1:1 CHCl₃/AcOEt adding EtOH at a 1%-10% gradient.

400 mg of 3-(1′-piperidinyl-iminomethyl)-16,17,18,19,28,29-hexahydrorifamycin S are obtained with Rf=0.8 on TLC in 1:1 CHCl₃/AcOH with 10% EtOH.

¹H-NMR (CDCl₃) 400 MHz: 0.2 (d, 3H); 0.4 (d, 3H); 0.7 (d, 3H); 1.06 (d, 3H); 1.2 (d, 3H); 1.2-1.8 (m, total 6H); 1.8 (m, 6H); 1.8 (s, 3H); 2.0 (s, 3H); 2.3 (s, 3H); 3.0-3.8 (m, total 6H); 3.2 (m, 4H); 5.01 (d, 1H); 7.8 (s, 1H); 11.0 (s, 1H).

### Example 8

### 3-(1′-piperidinyl-iminomethyl)-16,17,18,19,28,29-hexahydrorifamycin SV

350 mg of 3-(1′-piperidinyl-iminomethyl)-16,17,18,19,28,29-hexahydrorifamycin S synthesized as described in example 7 are dissolved in a water-miscible organic solvent (e.g. MeOH or acetone) to which an aqueous 15% ascorbic acid solution is then added. The reaction mixture is concentrated under vacuum, the reduction product is extracted with CHCl₃; the organic phase is washed with water to neutral pH then with saturated NaCl solution, dried over anhydrous Na₂SO₄, filtered and concentrated to dryness.

The product is purified using a column packed with 25 g of 200-425 mesh silica in 1:1 CHCl₃/AcOEt and eluted with 1:1 CHCl₃/AcOEt adding EtOH at a 1%-10% gradient.

200 mg of 3-(1′-piperidinyl-iminomethyl)-16,17,18,19,28,29-hexahydrorifamycin SV are obtained with Rf=0.44 on TLC in 1:1 CHCl₃/AcOEt with 10% of EtOH.

¹H-NMR (CDCl₃) 400 MHz: −0.3 (d, 3H); 0.5 (d, 3H); 0.7 (d, 3H); 0.9 (d, 3H); 1.3 (d, 3H); 1.3-1.8 (m, total 6H); 1.7 (s, 3H); 2.0 (s, 3H); 2.2 (s, 3H); 2.9 (m, 1H); 3.05 (s, 3H); 3.3 (m, 4H); 3.4-3.8 (m, total 4H); 4.9 (d, 1H); 8.3 (s, 1H); 12.0 (s, 1H).

### Example 9

### 3-(4′-phenyl-1′-piperazinyl-iminomethyl)-16,17,18,19,28,29-hexahydrorifamycin S

2.0 g of 16,17,18,19,28,29-hexahydrorifamycin S or SV, prepared as described in example 1 are dissolved in 50 ml of THF; 1.27 ml of t-butylamine, 530 μl of 37% formaldehyde and 1.3 g of MnO₂ are then added. The reaction is allowed to proceed overnight under agitation at 50° C. It is filtered over celite to remove MnO₂ and concentrated. Water is added and the mixture extracted with CHCl₃; the organic phase is subsequently washed with saturated NaCl solution, dried over anhydrous Na₂SO₄, filtered and concentrated to dryness.

The crude reaction product is dissolved in 50 ml of THF and 504.4 mg of 1-amino-4-phenylpiperazine, prepared as described for the 1-amino-4-ethylpiperazine of example 5, are added. The reaction is left for 4-5 hours under agitation at ambient temperature and concentrated; water is added and the mixture extracted with CHCl₃; the organic phase is subsequently washed with saturated NaCl solution, dried over anhydrous Na₂SO₄, filtered and concentrated to dryness.

The product is purified using a column packed with 150 g of 200-425 mesh silica in 1:1 CHCl₃/AcOEt and eluted with 1:1 CHCl₃/AcOEt adding EtOH at a 1%-10% gradient.

200 mg of 3-(4′-phenyl-1′-piperazinyl-iminomethyl)-16,17,18,19,28,29-hexahydrorifamycin S are obtained with Rf=0.45 on TLC in 1:1 CHCl₃/AcOEt.

¹H-NMR (CDCl₃) 400 MHz: 0.245 (d, 3H); 0.53 (d, 3H); 0.7 (d, 3H); 0.95 (d, 3H); 1.2-1.8 (m, total 6H); 1.66 (d, 3H); 1.6 (s, 3H); 2.05 (s, 3H); 2.27 (s, 3H); 3.2 (m, 4H); 3.48 (m, 4H); 2.8 (m, 1H); 3.4-3.7 (m, total 4H); 5.01 (d, 3H); 6.8 (m, 3H); 7.3 (m, 2H); 7.84 (s, 1H); 10.8 (s, 1H).

### Example 10

### 3-(4′-phenyl-1′-piperazinyl-iminomethyl)-16,17,18,19,28,29-hexahydrorifamycin SV

280 mg of 3-(4′-phenyl-1′-piperazinyl-iminomethyl)-16,17,18,19,28,29-hexahydrorifamycin S synthesized as described in example 9 are dissolved in a water-miscible organic solvent (e.g. MeOH or acetone) to which an aqueous 15% ascorbic acid solution is then added. The reaction mixture is concentrated under vacuum, the reduction product is extracted with CHCl₃; the organic phase is washed with water to neutral pH then with saturated NaCl solution, dried over anhydrous Na₂SO₄, filtered and concentrated to dryness.

The product is purified using a column packed with 20 g of 200-425 mesh silica in 1:1 CHCl₃/AcOEt and eluted with 1:1 CHCl₃/AcOEt adding EtOH at a 1%-10% gradient.

100 mg of 3-(4′-phenyl-1′-piperazinyl-iminomethyl)-16,17,18,19,28,29-hexahydrorifamycin SV are obtained with Rf=0.83 on TLC in 1:1 CHCl₃/AcOEt with 10% of EtOH.

¹H-NMR (CDCl₃) 400 MHz: −0.22 (d, 3H); 0.45 (d, 3H); 0.62 (d, 3H); 0.9 (d, 3H); 1.25 (d, 3H); 1.2-1.65 (m, total 6H); 1.6 (s, 3H); 2.0 (s, 3H); 2.2 (s, 3H); 2.8 (m, 1H); 3.15 (s, 3H); 3.3-3.6 (m, total 4H); 3.4 (m, 4H); 4.98 (d, 1H); 6.9 (m, 3H); 7.3 (m, 2H); 8.4 (s, 1H); 12.2 (s, 1H).

### Example 11

### 3-[4′-(2-ethoxyethyl)-1′-piperazinyl-iminomethyl]-16,17,18,19,28,29-hexahydrorifamycin S

2.0 g of 16,17,18,19,28,29-hexahydrorifamycin S or SV, prepared as described in example 1 are dissolved in 50 ml of THF; 1.27 ml of t-butylamine, 530 μl of 37% formaldehyde and 1.3 g of MnO₂ are then added. The reaction is allowed to proceed overnight under agitation at 50° C. It is filtered over celite to remove MnO₂ and concentrated. Water is added and the mixture extracted with CHCl₃; the organic phase is subsequently washed with a saturated NaCl solution, dried over anhydrous Na₂SO₄, filtered and concentrated to dryness.

The crude reaction product is dissolved in 50 ml of THF and 950 mg of 1-amino-4-(2-ethoxyethyl)-piperazine, prepared as described for the 1-amino-4-ethylpiperazine of example 5, are added. The reaction is left for 4-5 hours under agitation at ambient temperature and concentrated; water is added and the mixture extracted with CHCl₃; the organic phase is subsequently washed with saturated NaCl solution, dried over anhydrous Na₂SO₄, filtered and concentrated to dryness.

The product is purified using a column packed with 150 g of 200-425 mesh silica in 1:1 CHCl₃/AcOEt and eluted with 1:1 CHCl₃/AcOEt adding EtOH at a 1%-10% gradient.

300 mg of 3-[4′-(2-ethoxyethyl)-1′-piperazinyl-iminomethyl]-16,17,18,19,28,29-hexahydrorifamycin S are obtained with Rf=0.43 on TLC in 1:1 CHCl₃/AcOEt with 10% of EtOH.

¹H-NMR (CDCl₃) 400 MHz: 0.24 (d, 3H); 0.4 (d, 3H); 0.64 (d, 3H); 0.84 (d, 3H); 1.14 (t, 3H); 1.28 (d, 3H); 1.64 (s, 3H); 1.0-1.6 (m, total 6H); 2.0 (s, 3H); 2.24 (s, 3H); 2.8 (m, 6H); 3.0 (s, 3H); 3.4 (m, 8H); 2.9-3.4 (m, total 5H); 5.0 (d, 1H); 7.8 (s, 1H); 10.8 (s, 1H).

### Example 12

### 3-[4′-(2-ethoxyethyl)-1′-piperazinyl-iminomethyl]-16,17,18,19,28,29-hexahydrorifamycin SV

200 mg of 3-[4′-(2-ethoxyethyl)-1′-piperazinyl-iminomethyl]-16,17,18,19,28,29-hexahydrorifamycin S synthesized as described in example 11 are dissolved in a water-miscible organic solvent (e.g. MeOH or acetone) to which an aqueous 15% ascorbic acid solution is then added. The reaction mixture is concentrated under vacuum, the reduction product is extracted with CHCl₃; the organic phase is washed with water to neutral pH then with saturated NaCl solution, dried over anhydrous Na₂SO₄, filtered and concentrated to dryness.

The product is purified using a column packed with 15 g of 200-425 mesh silica in 1:1 CHCl₃/AcOEt and eluted with 1:1 CHCl₃/AcOEt adding EtOH at a 1%-10% gradient.

110 mg of 3-[4′-(2-ethoxyethyl)-1′-piperazinyl-iminomethyl]-16,17,18,19,28,29-hexahydrorifamycin SV are obtained with Rf=0.51 on TLC in 1:1 CHCl₃/AcOEt with 10% of EtOH.

¹H-NMR (CDCl₃) 400 MHz: −0.3 (d, 3H); 0.5 (d, 3H); 0.7 (d, 3H); 0.9 (d, 3H); 1.22 (t, 3H); 1.3 (d, 2H); 1.4-1.6 (m, total 6H); 1.7 (s, 3H); 2.02 (s, 3H); 2.2 (s, 3H); 2.75 (m, 4H); 2.95 (m, 1H); 3.05 (s, 3H); 3.22 (m, 4H); 3.2-3.8 (m, total 10H); 4.98 (d, 1H); 8.2 (s, 1H); 12.0 (s, 1H).

### Example 13

### 3-(1′-morpholinyl-iminomethyl)-16,17,18,19,28,29-hexahydrorifamycin S

2.0 g of 16,17,18,19,28,29-hexahydrorifamycin S or SV, prepared as described in example 1 are dissolved in 50 ml of THF; 1.27 ml of t-butylamine, 530 μl of 37% formaldehyde and 1.3 g of MnO₂ are then added. The reaction is allowed to proceed overnight under agitation at 50° C. It is filtered over celite to remove MnO₂ and concentrated. Water is added and the mixture extracted with CHCl₃; the organic phase is subsequently washed with a saturated NaCl solution, dried over anhydrous Na₂SO₄, filtered and concentrated to dryness.

The crude reaction product is dissolved in 50 ml of THF and 291 ml of 4-aminomorpholine (commercial) are added. The reaction is left for 4-5 hours under agitation at ambient temperature and concentrated; water is added and the mixture extracted with CHCl₃; the organic phase is subsequently washed with saturated NaCl solution, dried over anhydrous Na₂SO₄, filtered and dried.

The product is purified using a column packed with 150 g of 200-425 mesh silica in 1:1 CHCl₃/AcOEt and eluted with 1:1 CHCl₃/AcOEt adding EtOH at a 1%-10% gradient.

200 mg of 3-(1-morpholinyl-iminomethyl)-16,17,18,19,28,29-hexahydrorifamycin S are obtained with Rf=0.75 on TLC in 1:1 CHCl₃/AcOEt with 10% of EtOH.

¹H-NMR (CDCl₃) 400 MHz: 0.3 (d, 3H); 0.5 (d, 3H); 0.7 (d, 3H); 1.0 (d, 3H); 1.3 (d, 3H); 1.7 (s, 3H); 1.2-1.6 (m, total 6H); 2.0 (s, 3H); 2.3 (s, 3H); 2.8 (m, 1H); 3.0 (s, 3H); 3.2 (m, 4H); 3.2-3.6 (m, total 4H); 5.0 (d, 1H); 7.9 (s, 1H); 10.9 (s, 1H).

### Example 14

### 3-(1% morpholinyl-iminomethyl)-16,17,18,19,28,29-hexahydrorifamycin SV

250 mg of 3-(t-morpholinyl-iminomethyl)-16,17,18,19,28,29-hexahydrorifamycin S synthesized as described in example 13 are dissolved in a water-miscible organic solvent (e.g. MeOH or acetone) to which an aqueous 15% ascorbic acid solution is then added. The reaction mixture is concentrated under vacuum, the reduction product is extracted with CHCl₃; the organic phase is washed with water to neutral pH then with saturated NaCl solution, dried over anhydrous Na₂SO₄, filtered and concentrated to dryness.

The product is purified using a column packed with 18 g of 200-425 mesh silica in 1:1 CHCl₃/AcOEt and eluted with 1:1 CHCl₃/AcOEt adding EtOH at a 1%-10% gradient.

150 mg of 3-(t-morpholinyl-iminomethyl)-16,17,18,19,28,29-hexahydrorifamycin SV are obtained with Rf=0.74 on TLC in 1:1 CHCl₃/AcOEt with 10% of EtOH.

¹H-NMR (CDCl₃) 400 MHz: −0.3 (d, 3H); 0.5 (d, 3H); 0.9 (d, 3H); 1.3 (d, 3H); 1.2-1.6 (m, total 6H); 1.7 (s, 3H); 2.0 (s, 3H); 2.2 (s, 3H); 2.9 (m, 1H); 3.1 (s, 3H); 3.2 (m, 4H); 3.8-3.85 (m, total 3H); 3.9 (m, 4H); 5.0 (d, 1H); 8.4 (s, 1H); 12.2 (s, 1H).

### Example 15

### 3-(4′-isopropyl-1′-piperazinyl-iminomethyl)-16,17,18,19,28,29-hexahydrorifamycin SV

2.0 g of 16,17,18,19,28,29-hexahydrorifamycin S or SV, prepared as described in example 1, are dissolved in 50 ml of THF; 1.27 ml of t-butylamine, 530 μl of 37% formaldehyde and 1.3 g of MnO₂ are then added. The reaction is allowed to proceed overnight under agitation at 50° C. It is filtered over celite to remove MnO₂ and concentrated. Water is added and the mixture extracted with CHCl₃; the organic phase is subsequently washed with saturated NaCl solution, dried over anhydrous Na₂SO₄, filtered and concentrated to dryness.

The crude reaction product is dissolved in 50 ml of THF and 783 mg of 1-amino-4-isopropylpiperazine, prepared as described in example 5 for 1-amino-4-ethylpiperazine. The reaction is left for 4-5 hours under agitation at ambient temperature and concentrated; water is added and the mixture extracted with CHCl₃; the organic phase is subsequently washed with saturated NaCl solution, dried over anhydrous Na₂SO₄, filtered and dried.

Monitoring by TLC shows the presence of the final product in both the S and SV forms, in equivalent quantities; the mixture is then treated with a 15% ascorbic acid solution to obtain the SV form exclusively.

The product is purified using a column packed with 150 g of 200-425 mesh silica in 1:1 CHCl₃/AcOEt and eluted with 1:1 CHCl₃/AcOEt adding EtOH at a 1%-10% gradient.

300 mg of 3-(4′-isopropyl-1′-piperazinyl-iminomethyl)-16,17,18,19,28,29-hexahydrorifamycin SV are obtained with Rf=0.25 on TLC in 1:1 CHCl₃/AcOEt with 10% of EtOH.

¹H-NMR (CDCl₃) 400 MHz: −0.24 (d, 3H); 0.5 (d, 3H); 0.7 (d, 3H); 0.98 (d, 3H); 1.1 (d, 3H); 1.3 (dd, 6H); 1.6 (m, 5H); 1.62 (s, 3H); 2.0 (s, 3H); 2.2 (s, 3H); 2.8 (m, 5H); 3.1 (s, 3H); 3.2 (m, 4H); 3.3-3.5 (m, total 3H); 4.9 (d, 1H); 8.2 (s, 1H); 12.2 (s, 1H).

### Example 16

### 3-(4′-isopropyl-1′-piperazinyl-iminomethyl)-16,17,18,19,28,29-hexahydrorifamycin S

The product 3-(4′-isopropyl-1′-piperazinyl-iminomethyl)-16,17,18,19,28,29-hexahydrorifamycin SV, synthesized as described in example 15, is dissolved in CHCl₃ and shaken with an aqueous 33% potassium ferricyanide solution. The oxidation product is extracted with CHCl₃, the organic phase is washed with saturated NaCl solution, dried over anhydrous Na₂SO₄, filtered and concentrated to dryness.

The product 3-(4′-isopropyl-1′-piperazinyl-iminomethyl)-16,17,18,19,28,29-hexahydrorifamycin S has a Rf=0.3 on TLC in 1:1 CHCl₃/AcOEt with 10% of EtOH.

¹H-NMR (CDCl₃) 400 MHz: 0.2 (d, 3H); 0.4 (d, 3H); 0.6 (d, 3H); 0.8 (d, 3H); 1.0 (d, 3H); 1.3 (m, 6H); 1.6 (m, 9H); 2.0 (s, 3H); 2.2 (s, 3H); 2.6 (m, 4H); 3.2 (s, 1H); 3.3-3.7 (m, total 7H); 5.0 (d, 1H); 7.8 (s, 1H); 10.8 (s, 1H).

### Example 17

### 3-(1′-thiomorpholinyl-iminomethyl)-16,17,18,19,28,29-hexahydrorifamycin S

2.0 g of 16,17,18,19,28,29-hexahydrorifamycin S or SV, prepared as described in example 1 are dissolved in 50 ml of THF; 1.27 ml of t-butylamine, 530 μl of 37% formaldehyde and 1.3 mg of MnO₂ are then added. The reaction is allowed to proceed overnight under agitation at 50° C. It is filtered over celite to remove MnO₂ and concentrated. Water is added and the mixture extracted with CHCl₃; the organic phase is subsequently washed with saturated NaCl solution, dried over anhydrous Na₂SO₄, filtered and concentrated to dryness.

The crude reaction product is dissolved in 50 ml of THF and 648 mg of 4-amino-thiomorpholine, prepared as described in example 5 for 1-amino-4-ethylpiperazine. The reaction is left for 4-5 hours under agitation at ambient temperature and concentrated; water is added and the mixture extracted with CHCl₃; the organic phase is subsequently washed with saturated NaCl solution, dried over anhydrous Na₂SO₄, filtered and concentrated to dryness. The product is purified using a column packed with 150 g of 200-425 mesh silica in 1:1 CHCl₃/AcOEt and eluted with 1:1 CHCl₃/AcOEt adding EtOH at a 1%-10% gradient.

480 mg of 3-(1′-thiomorpholinyl-iminomethyl)-16,17,18,19,28,29-hexahydrorifamycin S are obtained with Rf=0.48 on TLC in 1:1 CHCl₃/AcOEt with 10% of EtOH.

¹H-NMR (CDCl₃) 400 MHz: 0.3 (d, 3H); 0.5 (d, 3H); 0.6 (d, 3H); 1.0 (d, 3H); 1.4 (d, 3H); 1.4-1.7 (m, total 5H); 1.75 (s, 3H); 2.01 (s, 3H); 2.2 (s, 3H); 2.8 (m, 4H); 3.2 (s, 3H); 3.4-3.8 (m, total 7H); 5.0 (d, 1H); 7.8 (s, 1H); 10.8 (s, 1H).

### Example 18

### 3-(1′-thiomorpholinyl-iminomethyl)-16,17,18,19,28,29-hexahydrorifamycin SV

250 mg of 3-(t-thiomorpholinyl-iminomethyl)-16,17,18,19,28,29-hexahydrorifamycin S synthesized as described in example 17, are dissolved in a water-miscible organic solvent (e.g. MeOH or acetone) to which an aqueous 15% ascorbic acid solution is then added. The reaction mixture is concentrated under vacuum, the reduction product is extracted with CHCl₃; the organic phase is washed with water to neutral pH, then with saturated NaCl solution, dried over anhydrous Na₂SO₄, filtered and dried.

The product is purified using a column packed with 20 g of 200-425 mesh silica in 1:1 CHCl₃/AcOEt and eluted with 1:1 CHCl₃/AcOEt adding EtOH at a 1%-10% gradient.

150 mg of 3-(t-thiomorpholinyl-iminomethyl)-16,17,18,19,28,29-hexahydrorifamycin S are obtained with Rf=0.8 on TLC in 1:1 CHCl₃/AcOEt with 10% of EtOH.

¹H-NMR (CDCl₃) 400 MHz: −0.6 (d, 3H); 0.2 (d, 3H); 0.4 (d, 3H); 0.7 (d, 3H); 0.9 (d, 3H); 1.1-1.4 (m, total 6H); 1.5 (s, 3H); 1.8 (s, 3H); 1.9 (s, 3H); 2.6 (m, 4H); 2.8 (s, 3H); 3.2-3.5 (m, total 7H); 4.8 (d, 1H); 8 (s, 1H); 11.8 (s, 1H).

### Example 19

### 3-(4′-cyclohexyl-1′-piperazinyl-iminomethyl)-16,17,18,19,28,29-hexahydrorifamycin S

2.0 g of 16,17,18,19,28,29-hexahydrorifamycin S or SV, prepared as described in example 1 are dissolved in 50 ml of THF; 1.27 ml of t-butylamine, 530 μl of 37% formaldehyde and 1.3 g of MnO₂ are then added. The reaction is allowed to proceed overnight under agitation at 50° C. It is filtered over celite to remove MnO₂ and concentrated. Water is added and the mixture extracted with CHCl₃; the organic phase is subsequently washed with saturated NaCl solution, dried over anhydrous Na₂SO₄, filtered and concentrated to dryness.

The crude reaction product is dissolved in 50 ml of THF and 1.50 ml of 1-amino-4-cyclohexylpiperazine, prepared as described in example 5 for 1-amino-4-ethylpiperazine, are added. The reaction is left for 4-5 hours under agitation at ambient temperature and concentrated; water is added and the mixture extracted with CHCl₃; the organic phase is subsequently washed with saturated NaCl solution, dried over anhydrous Na₂SO₄, filtered and dried.

The product is purified using a column packed with 150 g of 200-425 mesh silica in 1:1 CHCl₃/AcOEt and eluted with 1:1 CHCl₃/AcOEt adding EtOH at a 1%-10% gradient.

432 mg of 3-(4′-cyclohexyl-1′-piperazinyl-iminomethyl)-16,17,18,19,28,29-hexahydrorifamycin S are obtained with Rf=0.71 on TLC in 1:1 CHCl₃/AcOEt.

¹H-NMR (CDCl₃) 400 MHz: 0.3 (d, 3H); 0.4 (d, 3H); 0.6 (d, 3H); 0.9 (d, 3H); 1.2 (m, 6H); 1.25 (d, 3H); 1.6 (m, 5H); 1.62 (s, 3H); 1.2-1.6 (m, total 5H); 2.0 (s, 3H); 2.2 (s, 3H); 2.8 (m, 4H); 3.2 (s, 3H); 3.25-3.6 (m, total 3H); 4.9 (d, 1H); 7.8 (s, 1H); 10.8 (s, 1H).

### Example 20

### 3-(4′-cyclohexyl-1′-piperazinyl-iminomethyl)-16,17,18,19,28,29-hexahydrorifamycin SV

250 mg of 3-(4′-cyclohexyl-t-piperazinyl-iminomethyl)-16,17,18,19,28,29-hexahydrorifamycin S synthesized as described in example 19 are dissolved in a water-miscible organic solvent (e.g. MeOH or acetone) to which an aqueous 15% ascorbic acid solution is then added. The reaction mixture is concentrated under vacuum, the reduction product is extracted with CHCl₃; the organic phase is washed with water to neutral pH then with saturated NaCl solution, dried over anhydrous Na₂SO₄, filtered and concentrated to dryness.

The product is purified using a column packed with 20 g of 200-425 mesh silica in 1:1 CHCl₃/AcOEt and eluted with 1:1 CHCl₃/AcOEt adding EtOH at a 1%-10% gradient.

180 mg of 3-(4′-cyclohexyl-t-piperazinyl-iminomethyl)-16,17,18,19,28,29-hexahydrorifamycin SV are obtained with Rf=0.66 on TLC in 1:1 CHCl₃/AcOEt with 10% of EtOH.

¹H-NMR (CDCl₃) 400 MHz: −0.3 (d, 3H); 0.5 (d, 3H); 0.7 (d, 3H); 1.0 (d, 3H); 1.22 (d, 3H); 1.6 (s, 3H); 1.05-1.8 (m, total 10H); 2.0 (s, 3H); 2.2 (s, 3H); 2.8 (m, 4H); 2.85 (m, 1H); 3.1 (s, 3H); 3.2 (m, 4H); 3.4-3.6 (m, total 4H); 4.9 (d, 1H); 8.1 (s, 1H); 12.1 (s, 1H).

### Example 21

### 3-(4′-methyl-1′-piperazinyl-iminomethyl)-16,17,18,19,28,29-hexahydro-25-desacetyl-rifamycin SV

1.0 g (1.4 mmol) of rifamycin S are added under agitation to a solution of 20 g of KOH in 200 ml of EtOH, cooled with an ice bath. After 3 hours citric acid is added to neutral pH and the EtOH is eliminated under reduced pressure. The residue is taken up with CHCl₃ and the organic phase is washed first with H₂O then a saturated NaCl solution. The organic phase is dried over Na₂SO₄, filtered and evaporated. By means of TLC in 1:1 CHCl₃/AcOEt a yellow coloured spot appears relative to the product 25 O-desacetyl rifamycin S with Rf=0.30. The product is purified using a 70-230 mesh silica column (80 g) eluting with a 9:1 CH₂Cl₂/MeOH mixture. 0.8 g of compound 1 are obtained.

0.8 g of compound 1 dissolved in 50 ml of EtOH are hydrogenated for 6 hours under ordinary pressure and temperature in the presence of 100 mg of PtO₂. The mixture is then filtered over celite and evaporated to dryness under vacuum. Thus, 25-O-desacetyl 16,17,18,19,28,29 hexahydrorifamycin SV is obtained (Rf=0.3 on TLC in 9:1 CHCl₃:MeOH). 8 ml of 0.3 M NaNO₂ are added to the residue and 2M HCl until acidic pH is achieved. Water is added and the mixture extracted with CHCl₃; the organic phase is washed with saturated NaCl solution, dried over anhydrous Na₂SO₄, filtered and concentrated to dryness. 0.5 g of 25-O-desacetyl 16,17,18,19,28,29 hexahydrorifamycin S are obtained (Rf=0.7 on TLC in 9:1 CHCl₃/MeOH).

0.3 ml of t-butylamine, 112 μl of 37% formaldehyde and 270 mg of MnO₂ are added to a solution of 0.4 g of 25-O-desacetyl 16,17,18,19,28,29-hexahydrorifamycin S or SV, obtained as aforedescribed, dissolved in 10 ml of THF. The reaction is allowed to proceed overnight under agitation at 50° C. It is filtered over celite to remove MnO₂ and concentrated under vacuum. Water is added and the mixture extracted with CHCl₃; the organic phase is washed with a saturated NaCl solution, dried over anhydrous Na₂SO₄, filtered and evaporated to dryness.

The crude reaction product is dissolved in 10 ml of THF and 150 μl of 1-amino-4-methylpiperazine are added at ambient temperature and under agitation. The reaction continues to proceed for 4-5 hours under agitation. The mixture is then concentrated under vacuum, water is added and the mixture extracted with CHCl₃. The organic phase is washed with a saturated NaCl solution, dried over anhydrous Na₂SO₄, filtered and evaporated to dryness. The product is purified using a column of 70-230 mesh silica (50 g) eluting with a 95:5 CHCl₃/MeOH mixture. 43 g of 3-(4′-methyl-1′-piperazinyl-iminomethyl)-16,17,18,19,28,29-hexahydro-25-desacetyl-rifamycin SV are obtained (Rf=0.30 on TLC in 9:1 CHCl₃/MeOH).

¹H-NMR (CDCl₃) 300 MHz: −0.02 (d, 3H), 0.2 (d, 3H), 0.6 (d, 3H), 0.9 (d, 3H), 1.7 (s, 3H), 2.2 (s, 3H), 2.2 (s, 3H), 3.27 (s, 3H), 5.16 (d, 1H).

### Example 22

### 3-[4′-(2-hydroxyethyl)-1′-piperazinyl-iminomethyl]-16,17,18,19,28,29-hexahydrorifamycin SV

0.4 ml of t-butylamine, 150 μl of 37% formaldehyde and 300 mg of MnO₂ are added, under agitation and at ambient temperature, to a solution of 0.5 g of 16,17,18,19,28,29-hexahydrorifamycin S or SV, prepared as described in example 1, dissolved in 10 ml of THF. The reaction is allowed to proceed overnight under agitation at 50° C. It is then filtered over celite to remove MnO₂ and concentrated under vacuum. Water is added and the mixture extracted with CHCl₃; the organic phase is washed with a saturated NaCl solution, dried over anhydrous Na₂SO₄, filtered and concentrated to dryness.

The crude reaction product is dissolved in 10 ml of THF and 0.2 g of 1-amino-4-(2-hydroxyethyl)-piperazine are added at ambient temperature and under agitation. Agitation is continued for 4-5 hours at ambient temperature. The mixture is then concentrated under vacuum, water is added and the mixture extracted with CHCl₃. The organic phase is washed with a saturated NaCl solution, dried over anhydrous Na₂SO₄, filtered and concentrated to dryness. The product is purified using a column of 70-230 mesh silica (50 g) eluting with a 95:5 CHCl₃/MeOH mixture. 55 mg of 3-[4′-(2-hydroxyethyl)-1′-piperazinyl-iminomethyl)-16,17,18,19,28,29-hexahydrorifamycin SV are obtained (Rf=0.5 on TLC in 9:1 CHCl₃/MeOH).

¹H-NMR (CDCl₃) 300 MHz: −0.3 (d, 3H), 0.4 (d, 3H), 0.6 (d, 3H), 0.9 (d, 3H), 1.2 (d, 3H), 1.7 (s, 3H), 2.0 (s, 3H), 2.2 (s, 3H), 3.0 (s, 3H), 4.9 (d, 1H).

### Example 23

### 3-morpholine-21,23 O-isopropylidenerifamycin S

40 ml of dioxane and 2.37 ml of morpholine diluted in 5 ml of dioxane are added to 2.0 g of 21,23 O-isopropylidenerifamycin S prepared as described in example 3. The reaction is allowed to proceed overnight under agitation at ambient temperature. It is neutralized with an aqueous 10% citric acid solution. Water is added and the mixture extracted with AcOEt. The organic phase is washed with saturated NaCl solution, dried over anhydrous Na₂SO₄, filtered and concentrated to dryness. The product is purified using a column with 30 g of 200-425 mesh silica in 100% CH₂Cl₂ and after loading at gradient with AcOEt to 80:20.

400 mg of 3-morpholine-21,23 O-isopropylidenerifamycin S are obtained with Rf=0.63 on TLC in 85:15 CHCl₃/AcOEt

¹H-NMR (CDCl₃) 400 MHz: 0.4 (d, 3H); 0.6 (d, 3H); 0.7 (d, 3H); 0.8 (d, 3H); 1.2 (d, 3H); 1.2 (s, 3H); 1.7 (s, 3H); 1.68-1.7 (m, total 3H); 1.9 (s, 3H); 2.05 (s, 3H); 2.0 (m, 1H); 22.2 (s, 3H); 8 (s, 3H); 3.2-4.27 (m, total 11H); 4.8 (d, 1H); 5.1 (m, 1H); 5.9 (d, 1H); 6.0-6.2 (m, 3H); 7.8 (1H, NH); 13.17 (s, 1H).

### Example 24

### 3-morpholine-21,23-O-isopropylidenerifamycin SV

150 mg of 3-morpholine-21,23-O-isopropylidenerifamycin S synthesized as described in example 23 are dissolved in a water miscible organic solvent (e.g. MeOH or acetone) and a 15% ascorbic acid solution is added thereto. The reaction mixture is concentrated under vacuum, the reduction product is extracted with CHCl₃; the organic phase is washed with water to neutral pH, then with saturated NaCl solution, dried over anhydrous Na₂SO₄, filtered and concentrated to dryness.

The product is purified using a column packed with 18 g of 200-425 mesh silica in 1:1 CHCl₃/AcOEt and eluted with 1:1 CHCl₃/AcOEt adding EtOH at a 1%-10% gradient. 80 mg of 3-morpholine-21,23-O-isopropylidenerifamycin SV are obtained with Rf=0.8 on TLC in 85:15 CHCl₃/AcOEt.

¹H-NMR (CDCl₃) 400 MHz: −0.3 (d, 3H); 0.5 (d, 3H); 0.7 (d, 3H); 0.8 (d, 3H); 1.18 (d, 3H); 1.22 (s, 3H); 1.7 (s, 3H); 1.6-1.7 (m, total 3H); 1.9 (s, 3H); 2.0 (m, 1H); 2.1 (s, 3H); 2.2 (s, 3H); 2.8 (s, 3H); 3.1-4.27 (m, total 11H); 4.8 (d, 1H); 5.1 (m, 1H); 5.9 (d, 1H); 6.0-6.2 (m, 3H); 7.8 (1H, NH); 13.17 (s, 1H).

### Experimental Part

### 1. Antibacterial Activity

### Method:

To verify antibacterial activity, the plate diffusion method is used as described in the pharmacopeia:
A quantity of the sample is weighed and slowly dissolved in 25 ml of methanol R
A quantity of 0.05 M phosphate buffer at pH 7.0 is added so as to obtain the following concentrations: 50 μg/ml, 20 μg/ml, 10 μg/ml and 5 μg/ml
Plates were prepared with appropriate culture medium (agar thickness approximately 2-5 mm) inoculated with a suspension of Micrococcus luteus ATCC 10240, Staphylococcus aureus ATCC 6538 and Escherichia coli ATCC 8739.
0.1 ml of the different concentrations of each sample were dispensed into the wells formed in the agar plates
The plates were held for 1-2 hours at ambient temperature to allow diffusion of the sample and then incubated at 35-39° C. for not less than 18 hours.
The diameters of the inhibition halos of the compounds were measured with a gauge and compared with those of the reference substance, the results being expressed as percentages.

### Results:

Table A gives the percentage antibacterial activity towards Micrococcus luteus ATCC 10240 and the MIC values (Minimum Inhibitory Concentration) of the compounds relative to the following examples:

Example 23 (Sample SX1): 3-morpholine-21,23 O-isopropylidenerifamycin S, not pertaining to formula (I) of the invention, is inserted as the reference compound to indicate that the 21,23-O-isopropylidene derivatives of rifamycins S (unhydrogenated), when compared with rifamycin S (reference standard), maintain a certain antibacterial activity

Example 3 (Sample SX4): 3-(4′-methyl-1′-piperazinyl-iminomethyl)-21,23-O-isopropylidene-16,17,18,19,28,29-hexahydrorifamycin SV, compared with rifampicin (reference standard)

Example 1 (Sample SX5): 3-(4′-methyl-1′-piperazinyl-iminomethyl)-16,17,18,19,28,29-hexahydrorifamycin SV, compared with rifampicin (reference standard)

TABLE A

| Example | Sample | 50 μg/ml | 10 μg/ml | 5 μg/ml | MIC | |
|---|---|---|---|---|---|---|
| 23 | SX1 | 69% | 40% | 39% | <5 | μg/ml |
| 3 | SX4 | 0% | 0% | 0% | >50 | μg/ml |
| 1 | SX5 | 42% | 0% | 0% | 50-10 | μg/ml |

The results obtained with the Staphylococcus aureus ATCC 6538 plates are comparable to those of Micrococcus luteus while the results obtained with the Escherichia coli ATCC 8739 plates show a complete absence of antibacterial activity up to the maximum tested concentration of 50 μg/ml.

From the data obtained, it is deduced that the examples have an antibacterial activity ranging from substantially reduced to completely absent.

Table B gives the percentage antibacterial activity towards Micrococcus luteus ATCC 10240 compared with standard rifampicin, and the MIC values (Minimum Inhibitory Concentration) for the compounds relative to the following examples:

Example 6: 3-(4′-ethyl-1′-piperazinyl-iminomethyl)-16,17,18,19,28,29-hexahydrorifamycin SV

Example 12: 3-[4′-(2-ethoxyethyl)-1′-piperazinyl-iminomethyl]-16,17,18,19,28,29-hexahydrorifamycin SV

Example 15: 3-(4′-isopropyl-1′-piperazinyl-iminomethyl)-16,17,18,19,28,29-hexahydrorifamycin SV

TABLE B

| Example | 50 μg/ml | 20 μg/ml | 10 μg/ml | 5 μg/ml | MIC | |
|---|---|---|---|---|---|---|
| 6 | 39% | 0% | 0% | 0% | 50-20 | μg/ml |
| 12 | 0% | 0% | 0% | 0% | >50 | μg/ml |
| 15 | 0% | 0% | 0% | 0% | >50 | μg/ml |

From the data obtained, it is deduced that the antibacterial activity of the compounds relative to the examples in Table B is completely absent at a concentration of 20 μg/ml.

### 2. Activation of the PXR Receptor

### Method

The expression of cytochrome CYP3A4 was studied, employed as an enzyme test in engineered hepatocytes overexpressing for the human receptor PXR.

The assay consists of evaluating the ability of the new rifamycins, compared with positive controls consisting of 10 μM rifampicin and 10 μM Mevastatin and 0.1% DMSO as the negative control, to induce CYP3A4 gene expression in the cell line DPX2 (HepG2 line stably transfected with a vector containing human PXR and a vector hosting the PXRE enhancer upstream of the luciferase reporter gene). The activity of the new rifamycins is expressed as a ratio between luciferase activity in cells treated with the tested substance and that of the cells treated with DMSO. The viability and morphology of the cells are analyzed by optical microscopy.

### Results

The results obtained are illustrated in table 1 and in FIG. 1. From the results shown, it is deduced that the compounds relative to example 3 and example 1 according to the invention, are found to be from 1.5 to 3 times more active than the reference compound (rifampicin). The derivative SX1, comprising substitutions analogous to those described in the invention, but unhydrogenated in positions 16,17,18,19,28,29, is found to be decidedly less active.

TABLE 1
Effect of the compounds on increase in luciferase
activity mediated by CYP3A4 in cell line DPX2P29.

| | | Mean | Induction | Non | Weak to mod- | High | Cell |
|---|---|---|---|---|---|---|---|
| Compounds | | RLU | above DMSO | inducer* | erate inducer* | inducer* | appearance** |
| DMSO 0.1% | | 223 | 1.0 | X | | | 30% NR |
| Rifampicin | | 4657 | 22.2 | | | X | 30% MCM |
| 10 μM | | | | | | | |
| Mevastafin | | 758 | 3.4 | | X | | 30% MCM |
| 10 μM | | | | | | | |
| SX1 | 5 μM | 6537 | 29.3 | | | X | 30% MCM |
| | 10 μM | 7869 | 35.3 | | | X | 30% DC |
| | 20 μM | 4738 | 21.2 | | | X | 10% DC |
| | 40 μM | 396 | 1.8 | | X | | 0% DC |
| SX4 | 5 μM | 8052 | 36.1 | | | X | 30% MCM |
| | 10 μM | 10848 | 48.6 | | | X | 30% MCM |
| | 20 μM | 7125 | 32.0 | | | X | 20% MCM |
| | 40 μM | 4243 | 19.0 | | | X | 20% DC |
| SX5 | 5 μM | 8698 | 39.0 | | | X | 30% MCM |
| | 10 μM | 12069 | 54.1 | | | X | 30% MCM |
| | 20 μM | 12918 | 57.9 | | | X | 30% MCM |
| | 40 μM | 14636 | 65.6 | | | X | 30% MCM |

Cell confluence (%), nothing to observe (NR), dying cells (DC), modified cell morphology (MCM).

1-15. (canceled)

16. A compound of formula (I) 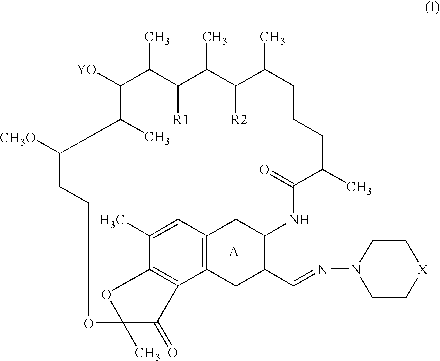
where
R₁ and R₂ are chosen from —OH or —OCH₃, otherwise R₁ and R₂ taken together form a —O—C(CH₃)₂—O— group,
the ring A is chosen from: 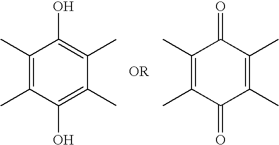
Y is chosen from H and —CO—CH₃,
X is chosen from CH₂, O, S, NH, NR₃, N—COR₃,
where R₃ denotes:
a) a linear or branched alkyl group having 1 to 9 carbon atoms,
b) a (CH₂)ₙ—R₄ chain, where n is 0 to 8 and R₄ is chosen from OH, NH₂, halogen, a cycloalkyl, aryl or heterocyclic group, with the proviso that when n is 1, R₄ is not phenyl, or
c) a (CH₂)ₘ—Z—(CH₂)ₙCH₃ chain, where m+n is 1 to 8, and Z denotes —O—, —S—, —NH—, —N(R₅), where R₅ is a linear or branched alkyl having 1 to 9 carbon atoms.

17. The compound according to claim 16, wherein X is NR₃.

18. The compound according to claim 17, wherein R₃ is:
a) a linear or branched alkyl having 1 to 4 carbon atoms,
b) a (CH₂)ₙ—R₄ chain, where n is 0 and R₄ is chosen from cyclohexyl, phenyl, piperidino, morpholino and thiomorpholino, or
c) a (CH₂)ₘ—Z—(CH₂)ₙ—CH₃ chain, where m+n is 1 to 5, and Z denotes —O—, —S—, —NH—, —NR₅—, where R₅ is a linear or branched alkyl having 1 to 9 carbon atoms.

19. The compound according to claim 16, chosen from:
3-(4′-methyl-1′-piperazinyl-iminomethyl)-16,17,18,19,28,29-hexahydrorifamycin SV
3-(4′-methyl-1′-piperazinyl-iminomethyl)-21,23-O-isopropylidene-16,17,18,19,28,29-hexahydrorifamycin SV
3-(4′-methyl-1′-piperazinyl-iminomethyl)-16,17,18,19,28,29-hexahydrorifamycin S
3-(4′-methyl-1′-piperazinyl-iminomethyl)-21,23-O-isopropylidene-16,17,18,19,28,29-hexahydrorifamycin S
3-(4′-ethyl-1′-piperazinyl-iminomethyl)-16,17,18,19,28,29-hexahydrorifamycin SV
3-(4′-ethyl-1′-piperazinyl-iminomethyl)-21,23-O-isopropylidene-16,17,18,19,28,29-hexahydrorifamycin SV
3-(4′-ethyl-1′-piperazinyl-iminomethyl)-16,17,18,19,28,29-hexahydrorifamycin S
3-(4′-ethyl-1′-piperazinyl-iminomethyl)-21,23-O-isopropylidene-16,17,18,19,28,29-hexahydrorifamycin S
3-(1′-piperidinyl-iminomethyl)-16,17,18,19,28,29-hexahydrorifamycin SV
3-(1′-piperidinyl-iminomethyl)-21,23-O-isopropylidene-16,17,18,19,28,29-hexahydrorifamycin SV
3-(1′-piperidinyl-iminomethyl)-16,17,18,19,28,29-hexahydrorifamycin S
3-(1′-piperidinyl-iminomethyl)-21,23-O-isopropylidene-16,17,18,19,28,29-hexahydrorifamycin S
3-(4′-phenyl-1′-piperazinyl-iminomethyl)-16,17,18,19,28,29-hexahydrorifamycin SV
3-(4′-phenyl-1′-piperazinyl-iminomethyl)-21,23-O-isopropylidene-16,17,18,19,28,29-hexahydrorifamycin SV
3-(4′-phenyl-1′-piperazinyl-iminomethyl)-16,17,18,19,28,29-hexahydro-rifamycin S
3-(4′-phenyl-1′-piperazinyl-iminomethyl)-21,23-O-isopropylidene-16,17,18,19,28,29-hexahydrorifamycin S
3-[4′-(2-ethoxyethyl)-1′-piperazinyl-iminomethyl]-16,17,18,19,28,29-hexahydrorifamycin SV
3-[4′-(2-ethoxyethyl)-1′-piperazinyl-iminomethyl]-21,23-O-isopropylidene-16,17,18,19,28,29-hexahydrorifamycin SV
3-[4′-(2-ethoxyethyl)-1′-piperazinyl-iminomethyl]-16,17,18,19,28,29-hexahydrorifamycin S
3-[4′-(2-ethoxyethyl)-1′-piperazinyl-iminomethyl]-21,23-O-isopropylidene-16,17,18,19,28,29-hexahydrorifamycin S
3-(1′-morpholinyl-iminomethyl)-16,17,18,19,28,29-hexahydrorifamycin SV
3-(1′-morpholinyl-iminomethyl)-21,23-O-isopropylidene-16,17,18,19,28,29-hexahydrorifamycin SV
3-(1′-morpholinyl-iminomethyl)-16,17,18,19,28,29-hexahydrorifamycin S
3-(1′-morpholinyl-iminomethyl)-21,23-O-isopropylidene-16,17,18,19,28,29-hexahydrorifamycin S
3-(1′-thiomorpholinyl-iminomethyl)-16,17,18,19,28,29-hexahydrorifamycin SV
3-(1′-thiomorpholinyl-iminomethyl)-21,23-O-isopropylidene-16,17,18,19,28,29-hexahydrorifamycin SV
3-(1′-thiomorpholinyl-iminomethyl)-16,17,18,19,28,29-hexahydrorifamycin S
3-(1′-thiomorpholinyl-iminomethyl)-21,23-O-isopropylidene-16,17,18,19,28,29-hexahydrorifamycin S
3-(4′-isopropyl-1′-piperazinyl-iminomethyl)-16,17,18,19,28,29-hexahydrorifamycin SV
3-(4′-isopropyl-1′-piperazinyl-iminomethyl)-21,23-O-isopropylidene-16,17,18,19,28,29-hexahydrorifamycin SV
3-(4′-isopropyl-1′-piperazinyl-iminomethyl)-16,17,18,19,28,29-hexahydrorifamycin S
3-(4′-isopropyl-1′-piperazinyl-iminomethyl)-21,23-O-isopropylidene-16,17,18,19,28,29-hexahydrorifamycin S
3-(4′-cyclohexyl-1′-piperazinyl-iminomethyl)-16,17,18,19,28,29-hexahydrorifamycin SV
3-(4′-cyclohexyl-1′-piperazinyl-iminomethyl)-21,23-O-isopropylidene-16,17,18,19,28,29-hexahydrorifamycin SV
3-(4′-cyclohexyl-1′-piperazinyl-iminomethyl)-16,17,18,19,28,29-hexahydrorifamycin S
3-(4′-cyclohexyl-1′-piperazinyl-iminomethyl)-21,23-O-isopropylidene-16,17,18,19,28,29-hexahydrorifamycin S
3-(4′-methyl-1′-piperazinyl-iminomethyl)-16,17,18,19,28,29-hexahydro-25-desacetylrifamycin SV
3-[4′-(2-hydroxyethyl)-1′-piperazinyl-iminomethyl]-16,17,18,19,28,29-hexahydrorifamycin SV

20. Method for the treatment of cholestasis and diseases related thereto comprising the administration of a compound of formula (I) 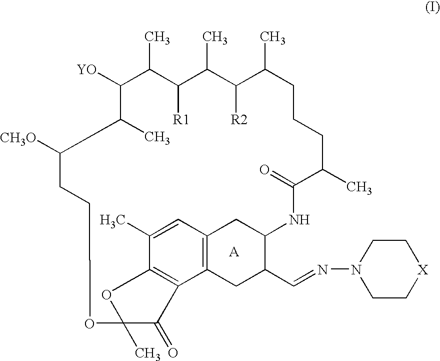
where
R₁ and R₂ are chosen from —OH or —OCH₃, otherwise R₁ and R₂ taken together form a —O—C(CH₃)₂—O— group,
the ring A is chosen from: 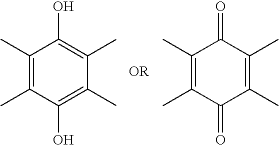
Y is chosen from H and —CO—CH₃,
X is chosen from CH₂, O, S, NH, NR₃, N—COR₃,
where R₃ denotes:
a) a linear or branched alkyl group having 1 to 9 carbon atoms,
b) a (CH₂)ₙ—R₄ chain, where n is 0 to 8, and R₄ is chosen from OH, NH₂, halogen, a cycloalkyl, aryl or heterocyclic group,
c) a (CH₂)ₘ—Z—(CH₂)ₙCH₃ chain, where m+n is 1 to 8, and Z denotes —O—, —S—, —NH—, —N(R₅), where R₅ is a linear or branched alkyl having 1 to 9 carbon atoms.

21. The method according to claim 20, wherein said diseases related to cholestasis are obstructive cholestasis, drug-induced cholestasis, Dubin-Johson Syndrome, sitosterolemia and in general all disorders of hepatobiliary transport.

22. A pharmaceutical composition comprising at least one compound of formula (I) according to claim 16 and at least one pharmaceutically acceptable excipient.

23. The pharmaceutical composition according to claim 22 comprising at least one dosage unit comprising 60 mg to 4000 mg of the compound of formula (I).

24. The pharmaceutical composition according to claim 22 in the form of a tablet, capsule, powder, granule, pill, liquid solution, suspension, emulsion, syrup, elixir, suppository, ointment, cream, lotion, gel, paste, transdermal formulation, medicated membrane or patch.

25. A process for preparing the compound of formula (I) according to claim 16 comprising the following steps:
(i) reduction of the double bonds in positions 16,17,18,19,28,29 of rifamycin S or SV; and
(ii) addition of the group 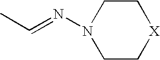
in position 3 of the product obtained in (i), where X has the meanings indicated in claim 16.

26. The process according to claim 25, wherein step (i) takes place by catalytic hydrogenation.

27. The process according to claim 25, wherein step (ii) takes place by treating the product of (i) with formaldehyde and a primary amine in the presence of an oxidizing agent, and then with a hydrazine of formula: 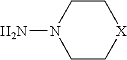
where X has the meanings as above defined in claim 16.

28. The process according to claim 25 for obtaining the compound of formula (I) having Y═H, comprising the use of C₂₅—O-desacetylrifamycin S or SV as the starting product.
